(19) United States
(12) Patent Application Publication
(10) Pub. No.: US 2026/0047936 A1
(43) Pub. Date: Feb. 19, 2026

(54) RUBBER KNEE REPLACEMENT APPARATUS AND METHOD FOR CUSTOMIZATION

(71) Applicant: David T. Domenici, Palm Desert, CA (US)

(72) Inventor: David T. Domenici, Palm Desert, CA (US)

(21) Appl. No.: 18/804,233

(22) Filed: Aug. 14, 2024

(51) Int. Cl. A61F 2/38; A61F 2/30

(52) U.S. Cl. CPC A61F 2/3859; A61F 2/30942; A61F 2/389; A61F 2002/30131; A61F 2310/00005

(57) ABSTRACT

A prosthetic knee replacement apparatus constructed of rubber avoids the disadvantages of metal prosthetics. The knee replacement apparatus includes a base member as a spacer between the upper and lower legs of a patient. A femoral portion extends upwardly from an upper surface of the base member and a tibial portion is coupled to a lower surface of the base member. The knee replacement apparatus may be constructed completely or substantially from a rubber material so as to reduce friction and degeneration. The knee replacement system may be constructed or even 3D printed after the leg bones of the patient are scanned.

### REFERENCE TO RELATED APPLICATIONS

This application is related to provisional Ser. No. 63/147,923 filed Feb. 10, 2021 and titled Rubber Knee Replacement System, which is incorporated in its entirety by reference.

### BACKGROUND OF THE INVENTION

This invention relates generally to prosthetic knee replacement devices and, more particularly, to a total knee replacement apparatus constructed substantially of rubber material that may be customized to a patient, flexible, and that avoids inflammation.

Many individuals suffer from knee pain. Knee pain may be caused by injuries and disease. Common injuries or conditions that may lead to increasing amounts of knee pain include ligament tears, patella tendinitis, arthritis, osteoporosis, dislocation, gout, and the like. Although the common recommendation includes rest, ice, compression, and the use of mobility devices, sometimes the pain becomes too intense as to be disruptive of a person's way of life or otherwise unbearable. In such an instance, a person may be a good candidate for total knee replacement in which the entire knee joint that receives the femur, fibula, and tibia bones. In other words, an otherwise healthy person may take on a prosthetic knee.

Unfortunately, most prosthetic knee joints are constructed entirely of metal which may cause more problems over time then were experienced prior to the surgery in the first place. Obviously, a metal joint may lack flexibility, may degenerate or rust, or may need to be replaced as technology may dictate.

Therefore, it would be desirable to have a prosthetic knee replacement joint constructed entirely or partially out of a rubber material that would provide the flexibility, comfort, and more financial desirability than existing prosthetics. Further, it would be desirable to have a prosthetic knee replacement joint in which ends of the three primary bones may be scanned prior to surgery and the prosthetic joint molded or even 3D printed specific to the patient.

### SUMMARY OF THE INVENTION

Natural rubber is a polymer found in plants that produce latex. The properties of rubber include high strength and the capability to be stretched many times without breaking. Natural rubber compounds are exceptionally flexible, good electrical insulators, and are resistant to many corrosive substances.

Therefore, a general object of this invention is to provide a knee replacement apparatus constructed entirely or substantially of a rubber material so as to be flexible, comfortable, and durable.

Another object of this invention is to provide a knee replacement apparatus, as aforesaid, that may be molded or 3D printed after medically scanning the bones of a patient to received the replacement apparatus. In other words, the knee replacement system may be customized to a particular patient.

Still another object of this invention is to provide a knee replacement apparatus, as aforesaid, that is cost effective to produce and to purchase by a patient.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a knee replacement apparatus according to a preferred embodiment of the present invention;

FIG. 2 is a sectional view taken along line 2-2 of FIG. 1;

FIG. 3 is an exploded view of the knee replacement apparatus as in FIG. 1;

FIG. 4 is a side view of the knee replacement apparatus of FIG. 2 removed from attachment to respective bones; and

FIG. 5 is an exploded view of the knee replacement apparatus of FIG. 2.

### DESCRIPTION OF THE PREFERRED EMBODIMENT

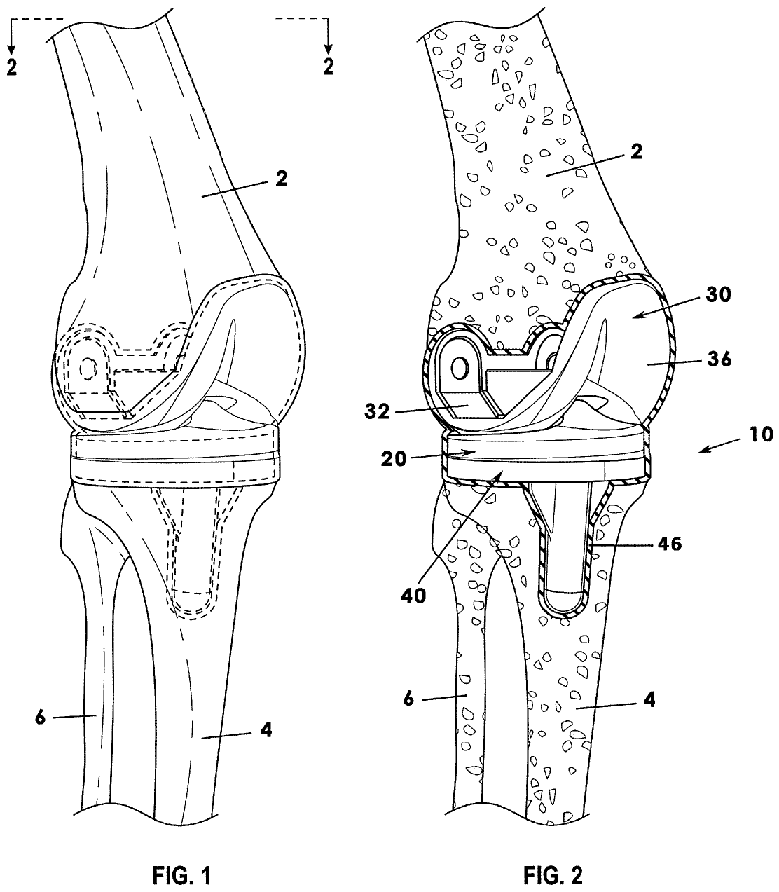

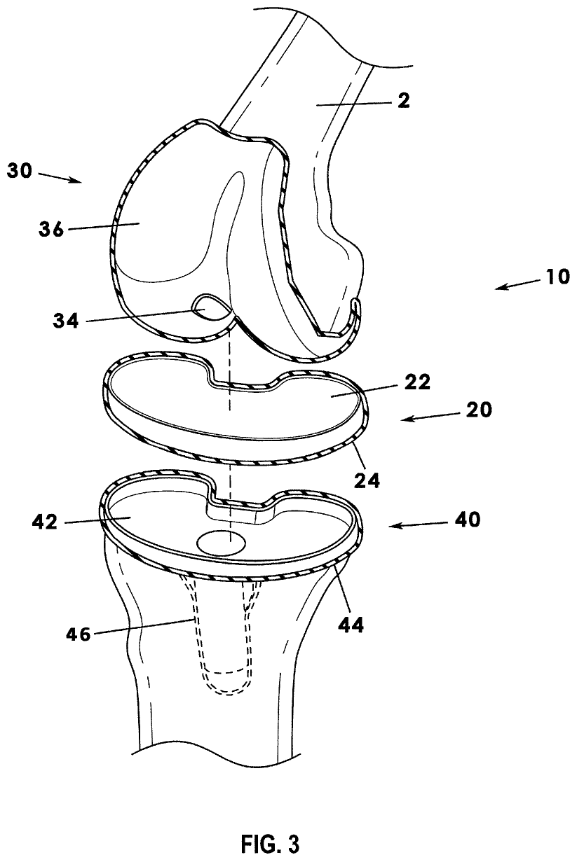

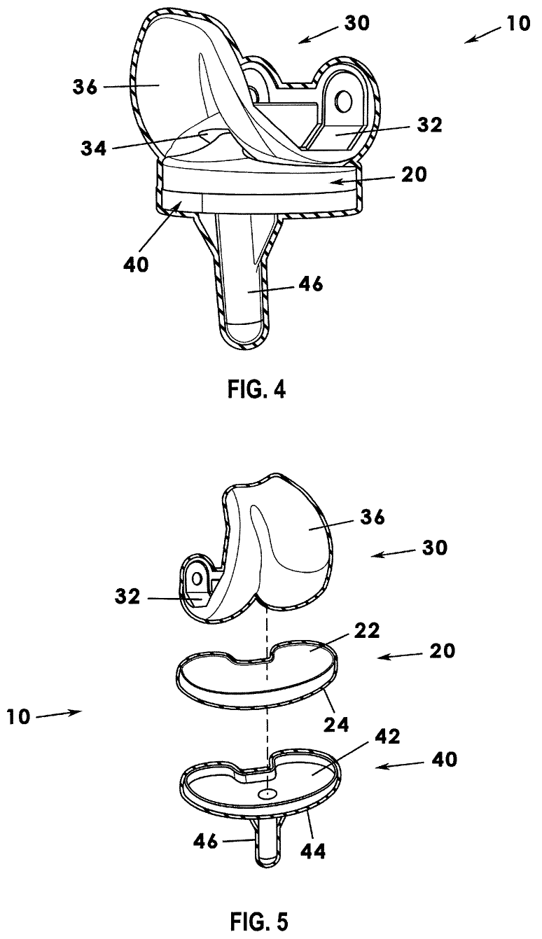

A rubber knee replacement apparatus according to a preferred embodiment of the present invention will now be described with reference to FIG. 1 to 5 of the accompanying drawings.

The knee replacement apparatus 10, also referred to as a knee replacement joint, includes a base member 20 having an upper surface 22 and a lower surface 24 opposite the upper surface 22 and defining a generally planar and laterally (side to side) symmetrical configuration. In an embodiment, the base member 20 may be referred to as a spacer as it is positioned intermediate a femoral portion 30 and tibial portion 40. In other words, the base member 20 is situated so as to keep the upper and lower leg bones separated and so the kneecap works efficiently. In an embodiment, the base member 20 may be constructed using a plastic material or, alternatively, a rubber material.

The knee replacement apparatus 10 also includes a femoral portion 30 coupled to and extending generally upwardly from the upper surface 22 of the base member 20 and is configured to receive a lower end of a femur 2 of the patient's upper leg. The femoral portion 30 may include a femur receiving section 32 having an upwardly concave configuration that is complementary to a lower end of a patient's femur 2. In other words, the femur receiving section 32 is a prosthesis configured to receive the end of a patient's femur 2 in a nesting arrangement. The femoral portion 30 has a bottom surface 36 extend upwardly from the upper surface 22 of the base member 20 and defines an aperture 34 for receiving the patella (kneecap), the patella being part of the kneecap joint enjoined to the respective ligaments within the femoral groove. Further, the dimensions and shape configuration of the femur receiving section 32 may be constructed after a scan is performed of the patient's femur bone, such as via an MRI or ultrasound scan, so that the nesting arrangement may be customized to the particular patient receiving the prosthetic knee replacement joint 10. More particularly, a medical scan according to a method associated with the present invention generates size and shape dimensional data that may be used in the manufacturing process so as to customize each and every patient who receives the knee replacement apparatus 10.

Preferably, the femoral portion 30 is constructed of rubber. Natural rubber is a polymer found in plants that produce latex. The properties of rubber include high strength and the capability to be stretched many times without breaking. Natural rubber compounds are exceptionally flexible, good electrical insulators, and are resistant to many corrosive substances. In an embodiment, the femoral portion 30 may include a construction using a rigid rubber material for strength.

The knee replacement apparatus 10 also includes a tibial portion 40 which may also be referred to as a tibial plate having a top surface 42 and a bottom surface 44 opposite the top surface. Further, the tibial portion 40 may include an anchor flange 46 extending downwardly from the bottom surface 44, the anchor being operative to be anchored into the tibia bone 4 during knee replacement surgery. In an embodiment, the top surface 42 of the tibial portion 40 may be fixedly attached to the lower surface 24 of the base member 20. Preferably, the tibial portion 40 and base member 20 include the same peripheral dimension so as to function as a single unit although the base member 20 may be constructed of a plastic or rubber material whereas the tibial portion 40 may be constructed of metal or, in an embodiment, of a rubber material. In an embodiment, the base member 20 and tibial portion 40 may have a unitary construction. In still other embodiments, the tibial portion 40 may be constructed partially of metal overlaid with rubber. Similar to the customization procedure of the femoral portion 30, a patient's tibia 4 and fibula 6 (lower leg bones) may first be scanned to determine its exact dimension and, then, the tibial portion 40 and base member 20 may be constructed based on the scan so that the tibial portion 40 may also be customized for the patient.

Generally speaking, the knee replacement apparatus 10 is constructed of a rubber material so as to avoid the disadvantages of a metal construction as described previously. For instance, having a rubber molded construction will reduce friction caused by functional metal parts, and in traditional knee replacement joint. It is understood, however, that at least the femoral portion 30 must include some degree of rigidity so as to maintain a proper orientation relative to the upper surface 22 of the base member 20. In this instance, the femoral portion 30 may be constructed of a rigid rubber material or constructed of a 3D resin coated with a layer of rubber material.

There is another polymer derived from a plant that provides enhanced strength characteristics, sufficient flexibility, and incredible biodegradability. Material is a hemp-based polymeric fiber. In other words, traditional petroleum-based plastics may increasingly be considered disadvantageous because they take a very long time to biodegrade. In an embodiment, therefore, all or part of the customized knee replacement apparatus 10 may be constructed using a hemp-fiber reinforced biopolymer. In such embodiments, the components of the knee replacement apparatus 10 may exhibit strength properties that are superior to conventional applicators.

It is understood that while certain forms of this invention have been illustrated and described, it is not limited thereto except insofar as such limitations are included in the following claims and allowable functional equivalents thereof.

1. A customized knee replacement apparatus, comprising:
a base member that includes an upper surface and a lower surface opposite said upper surface and having a planar and symmetrical configuration;
a femoral portion that includes a femur receiving section having an upwardly concave configuration that is constructed of rubber material according to a medical scan of a lower end of a patient's femur such that said femur receiving section receives the lower end of the patient's femur in a complementary nested relationship,
said femoral portion including a bottom surface coupled to the upper surface of said base member; and
a tibial portion that includes a top wall coupled to the lower surface of said base member and a bottom wall opposite the top wall, said tibial portion being constructed of rubber material according to a medical scan of upper ends of the patient's tibia and fibula bones, respectively.

2. The customized knee replacement apparatus as in claim 1, wherein said tibial portion has a peripheral configuration that is the same as a peripheral configuration of said base member.

3. The customized knee replacement apparatus as in claim 1, wherein:
said base member is constructed of rubber material; and
said tibial portion is at least partially constructed using metal.

4. The customized knee replacement apparatus as in claim 1, wherein the femoral portion is constructed of a rigid rubber material.

5. The customized knee replacement apparatus as in claim 1, wherein the femoral portion is constructed of a rapid prototype resin coated with rubber.

6. The customized knee replacement apparatus as in claim 1, wherein said medical scan is one of an MRI or ultrasound scan that is configured to generate size and shape data.

7. The customized knee replacement apparatus as in claim 1, wherein said tibial portion includes an anchor flange extending downwardly from the bottom wall and configured to interface with a patient's tibia.

8. The customized knee replacement apparatus as in claim 1, wherein said base member and said tibial portion have a unitary construction.

9. The customized knee replacement apparatus as in claim 1, wherein the bottom surface of the femoral portion defines an aperture configured for receiving the patient's patella.

10. The customized knee replacement apparatus as in claim 1, wherein the femur receiving section includes a pair of laterally adjacent lobes each having a generally U-shaped configuration.

11. A method for customizing a knee replacement apparatus, comprising:
providing a base member that includes an upper surface and a lower surface opposite said upper surface and that has a planar and symmetrical configuration;
medically scanning knee-ward ends of a patient's femur and tibia so as to generate dimensional data, respectively;
using said dimensional data, constructing a femoral portion using rubber material and that includes a femur receiving section having an upwardly concave configuration configured for receiving said knee-ward and of the patient's femur in a nested arrangement,
said femoral motion including a bottom surface coupled to the upper surface of the base member;
using said dimensional data, constructing a tibial portion using rubber material and that includes a top wall coupled to the lower surface of said base member and a bottom wall opposite the top wall.

12. The method as in claim 11, wherein:
the base member includes rubber; and said tibial portion includes rubber and metal.

13. The method as in claim 11, wherein the femoral portion has a rigid configuration that includes a rigid rubber material.

14. The method as in claim 11 wherein the femoral portion has a rigid configuration that includes a rapid prototype resin coated with rubber.

15. The method as in claim 11, wherein said dimensional data includes size and shape data.

16. The method as in claim 11, wherein said tibial portion includes an anchor flange extending downwardly from the bottom wall and configured to interface with a patient's tibia.

17. The method as in claim 11, wherein said base member and said tibial portion have a unitary construction.

18. The method as in claim 11, wherein the bottom surface of the femoral portion defines an aperture configured for receiving the patient's patella.

19. The method as in claim 11, wherein the femur receiving section includes a pair of laterally adjacent lobes each having a generally U-shaped configuration.

20. A customized knee replacement apparatus, comprising:
a base member that includes an upper surface and a lower surface opposite said upper surface and having a planar and symmetrical configuration;
a femoral portion that includes a femur receiving section having an upwardly concave configuration that is constructed of a hemp-based polymeric fiber according to a medical scan of a lower end of a patient's femur such that said femur receiving section receives the lower end of the patient's femur in a complementary nested relationship,
said femoral portion including a bottom surface coupled to the upper surface of said base member; and
a tibial portion that includes a top wall coupled to the lower surface of said base member and a bottom wall opposite the top wall, said tibial portion being constructed of a hemp-based polymeric fiber according to a medical scan of upper ends of the patient's tibia and fibula bones, respectively.
